(19) United States
(12) Patent Application Publication
(10) Pub. No.: US 2022/0338810 A1
(43) Pub. Date: Oct. 27, 2022

(54) EAR-WEARABLE DEVICE AND OPERATION THEREOF

(71) Applicant: NT LABS PVT LTD, Vadakara (IN)

(72) Inventor: Nitin VASANTH, Vadakara (IN)

(73) Assignee: NT LABS PVT LTD, Vadakara (IN)

(21) Appl. No.: 17/773,672

(22) PCT Filed: Nov. 4, 2020
(86) PCT No.: PCT/IB2020/060352

§ 371 Date: May 2, 2022

(30) Foreign Application Priority Data
Nov. 4, 2019 (IN) 201941040349

(51) Int. Cl. A61B 5/00; A61B 5/256; A61B 5/263; A61B 5/291; A61B 5/0245; A61B 5/024; A61B 5/01

(52) U.S. Cl. CPC A61B 5/6817; A61B 5/256; A61B 5/263; A61B 5/291; A61B 5/7435; A61B 5/0245; A61B 5/02438; A61B 5/01

(57) ABSTRACT

The present invention relates to an ear-wearable device (100) comprising: a plurality of neuro-buds (100a), each neuro-bud (100a) comprising: a housing (102), a hub (104) disposed in the housing (102), a plurality of springs (2, 2a-2h) disposed on the hub (104), and a biosensor electrode (1, 1a-1h) disposed on each spring (2, 2a-2h) and adapted to be in contact with an ear canal for detecting at least one physiological parameter of a user, wherein the plurality of springs (2, 2a-2h) are adapted to expand for extending the biosensor electrode (1, 1a-1h) to establish contact with the ear canal and to contract for retracting the biosensor electrode (1, 1a-1h) to break the contact; and a controller (100, 300) in communication with the biosensor electrode (1, 1a-1h) and adapted to: receive at least one value of the at least one physiological parameter detected by the biosensor electrode (1, 1a-1h), and generate health insights of the user based on the at least one physiological parameter.

### FIELD OF THE INVENTION

The present invention, in general, relates to the fields of a wearable biomedical device. More particularly, the invention relates to an ear-wearable biomedical device for monitoring various bio-signals and the operation thereof.

### BACKGROUND OF THE INVENTION

In recent decades, the anxieties of modern routine life, busy/hectic lifestyles have drastically elevated the probabilities of cardiovascular disease, mental illness or psychiatric disorder, hypotension/hypertension, etc. Studies reveal that an upsurge demands in embracing the technologies of biomedical-wearable and remote patient monitoring devices have extensively been driven by software and hardware constancy which have endorsed a range of devices/wearables to be adopted with reliance to guide or train an individual about his/her health, fitness and wellness, including prolonged condition supervision. Furthermore, certain conventional techniques have endeavored to elicit the feature of monitoring bio-signals like heart and brain activity by using scalp-wearable or ear-wearable devices. These wearable biomedical devices have led to revolutionizing the monitoring of diverse bio-signals with unobtrusive and high signal fidelity characteristics.

However, the drawbacks associated with such existing devices are that they may lack comfortability in wearing, or the device's specific usage in diagnosing one or two selective parameters only. The mechanism of bulky electrodes, being placed on the forehead and scalp and other body parts, makes existing wearable biomedical devices uncomfortable and less aesthetically pleasing for the users, especially for children. Another drawback associated with the prior existing technologies is that the sensor electrodes are fairly exposed which tends to capture and get interfered with other external Electromagnetic signal variations in the vicinity, rendering an inaccuracy in output readings.

Also, because the existing sensor electrodes are adapted to be placed loosely on the scalp, etc., they are more prone to motion artefacts and may determine subsequently inaccurate readings. Furthermore, the detected signals are usually used without appropriate processing of the bio-signals, which limits the information that can be extracted from it.

At least to address the aforesaid constraints, there lies a need for obviating aforesaid drawbacks plaguing the state of the art by providing a non-obstructive, and comfortable to wear apparatus for bio-signals monitoring, without hampering the signal fidelity.

More specifically, there lies a need for evolving an improved mechanism to advantageously address the requirements of at least an ear-wearable biomedical device comprising neuro-bud(s) and a method for efficiently-capturing the bio-signals during interfacing of the wearable biomedical device with the subject's body and utilizing the same for usage as a personalized physical and mental fitness coach. The present disclosure at least improves the wearability and portability of the ear-wearable biomedical device for unobstructed and simultaneous monitoring of different bio-signals.

### SUMMARY OF THE INVENTION

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present disclosure, an ear-wearable device is disclosed. The device includes a plurality of neuro-buds. Each neuro-bud includes a housing, a hub that is disposed in the housing, a plurality of springs disposed on the hub, and aa biosensor electrode disposed on each spring and adapted to be in contact with an ear canal for detecting at least one physiological parameter of a user. The plurality of springs is adapted to expand for extending the biosensor electrode to establish contact with the ear canal and to contract for retracting the biosensor electrode to break the contact. The ear-wearable device also includes a controller in communication with the biosensor electrode and adapted to receive at least one value of the at least one physiological parameter detected by the biosensor electrode and generate at least health insights of the user based on the at least one physiological parameter.

In another embodiment of the present disclosure, a neuro-bud of an ear-wearable device for detecting at least one physiological parameter of a user is disclosed. The neuro-bud includes a housing, a hub disposed in the housing, a plurality of springs disposed on the hub, and a biosensor electrode disposed on each spring and adapted to be in contact with an ear canal for detecting the at least one physiological parameter of the user. The plurality of springs is adapted to expand for extending the biosensor electrode for establishing contact with the ear canal and to contract for retracting the biosensor electrode for breaking the contact.

In another embodiment of the present disclosure, a neuro-bud of an ear-wearable device for detecting at least one physiological parameter of a user is disclosed. The neuro-bud includes a housing, a hub disposed in the housing, a plurality of flexible curved leaves disposed on the hub, and a biosensor electrode disposed on each spring and adapted to be in contact with an ear canal for detecting the at least one physiological parameter of the user. The plurality of flexible curved leaves is adapted to expand for extending the biosensor electrode to establish contact with the ear canal and to contract for retracting the biosensor electrode to break the contact.

In another embodiment of the present disclosure, a neuro-bud of an ear-wearable device for detecting at least one physiological parameter of a user is disclosed. The neuro-bud includes a housing, a hub that is disposed in the housing, a plurality of actuating members with spring-like properties on either side of the housing, a collapsible member assembly supported on the other end of the actuating members, an earbud, connecting arms with projected ends for holding the earbud, and a plurality of biosensor electrodes coupled to the earbud that are adapted to be in contact with an ear canal for detecting the at least one physiological parameter of the user. The collapsible member assembly is adapted to expand fur extending the biosensor electrode to establish contact with the ear canal and to retract for retracting the biosensor electrode to break the contact, based on the actuation of the actuating members.

In another embodiment of the present disclosure, an ear-wearable device for detecting at least one physiological parameter of a user is disclosed. The ear-wearable device includes a plurality of neuro-buds, where each neuro-bud further comprises a housing, a hub disposed of in the housing, and a plurality of actuating members with spring-like properties on either side of the housing. Each neuro-bud of the ear-wearable device further comprises an earbud, a collapsible member assembly supported on the other end of the actuating members, a plurality of biosensor electrodes coupled to the earbud, and connecting arms with projected ends to hold the earbud. The neuro-buds are adapted to be in contact with an ear canal for detecting the at least one physiological parameter of the user. The collapsible member assembly is adapted to expand for extending the biosensor electrode to establish contact with the ear canal and to retract for retracting the biosensor electrode to break the contact, based on the actuation of the actuating members. The ear-wearable device further comprises a controller in communication with the biosensor electrode. The controller is further adapted to: receive at least one value of the at least one physiological parameter detected by the biosensor electrode and generate at least health insights of the user based on the at least one physiological parameter.

To further clarify the advantages and features of the present inventive concepts, a more particular description of the inventive concepts will be rendered by reference to example embodiments thereof, which is illustrated in the appended drawings. It is appreciated that these drawings depict example embodiments of the inventive concepts and are therefore not to be considered limiting of its scope. The inventive concepts will be described and explained with additional specificity and detail with the accompanying drawings.

### BRIEF DESCRIPTION OF DRAWINGS

These and other features, aspects, and advantages of the present disclosure will become better understood when the following detailed description is read with reference to the accompanying drawings in which like characters represent like parts throughout the drawings, wherein:

FIG. 1(a) illustrates a schematic view of a wired ear-wearable device, in accordance with an embodiment of the present disclosure;

FIG. 1(b) illustrates a schematic view of a wireless ear-wearable device, in accordance with an embodiment of the present disclosure;

FIG. 1(e) illustrates a schematic view of a compact wireless ear-wearable device, in accordance with an embodiment of the present disclosure;

FIG. 2(a) illustrates a schematic view of a neuro-bud of the ear-wearable device depicting a plurality of springs in retracted mode, in accordance with an embodiment of the present disclosure;

FIG. 2(b) illustrates a schematic view of the neuro-bud depicting a plurality of springs in a released mode, in accordance with an embodiment of the present disclosure;

FIG. 2(c) illustrates a schematic perspective view of the neuro-bud, in accordance with an embodiment of the present disclosure;

FIG. 3 illustrates an exploded view of a controller of an ear-wearable device, in accordance with an embodiment of the present disclosure;

FIG. 4(a) illustrates a schematic view of an ear-wearable device depicting a plurality of curved leaves in a retracted mode, in accordance with an embodiment of the present disclosure;

FIG. 4(b) illustrates a schematic view of the neuro-bud depicting a plurality of curved leaves in a released mode, in accordance with an embodiment of the present disclosure:

FIG. 4(c) illustrates a schematic perspective view of the neuro-bud, in accordance with an embodiment of the present disclosure;

FIG. 5(a) illustrates a schematic view of a neuro-bud in a compressed state, in accordance with an embodiment of the present disclosure;

FIG. 5(b) illustrates a schematic view of the neuro-bud in an expanded state, in accordance with an embodiment of the present disclosure;

FIG. 5(c) illustrates a schematic perspective view of the neuro-buds, comprising collapsible member(s), in accordance with an embodiment of the present disclosure;

FIG. 6(a) illustrates an exploded view of the neuro-buds comprising collapsible member(s) as referred in FIG. 5, in accordance with an embodiment of the present disclosure;

FIG. 6(b) illustrates a schematic perspective view of the collapsible member of the neuro-bud, in accordance with an embodiment of the present disclosure;

FIG. 6(c) illustrates a cross-sectional view of the collapsible member and ear-bud of the neuro-bud, in accordance with an embodiment of the present disclosure; and

FIG. 7 illustrates a use case scenario of the ear-wearable device in communication with a User Equipment, in accordance with an embodiment of the present disclosure.

Further, skilled artisans will appreciate that elements in the drawings are illustrated for simplicity and may not have been drawn to scale. For example, the flow charts illustrate the method in terms of the most prominent operations involved to help improve understanding of aspects of the present inventive concepts. Furthermore, in terms of the construction of the device, one or more components of the device may have been represented in the drawings by conventional symbols, and the drawings may show only those specific details that are pertinent to understand the example embodiments of the present inventive concepts so as not to obscure the drawings with details that will be readily apparent to those of ordinary skill in the art having the benefit of the description herein.

### DETAILED DESCRIPTION OF FIGURES

For the purpose of promoting and understanding of the principles of the inventive concepts, reference will now be made to example embodiments illustrated in the drawings and specific language will be used to describe the same. It will nevertheless be understood that no limitation of the scope of the inventive concepts is thereby intended, such alterations and further modifications in the illustrated system, and such further applications of the principles of the inventive concepts as illustrated therein being contemplated as would normally occur to one skilled in the art to which the inventive concepts relate.

It will be understood by those skilled in the art that the foregoing general description and the following detailed description are explanatory of the present disclosure and are not intended to be restrictive thereof.

Reference throughout this specification to “an aspect”, “another aspect” or similar language means that a particular feature, structure, or characteristic described in connection with the example embodiments is included in at least one example embodiment of the present disclosure. Thus, appearances of the phrase “in example embodiments”, “in another example embodiment” and similar language throughout this specification may, but do not necessarily, all refer to the same example embodiments.

The terms “comprises”, “comprising”, or any other variations thereof, are intended to cover a non-exclusive inclusion, such that a process or method that comprises a list of operations does not include only those operations but may include other operations not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skilled in the art to which this disclosure belongs. The system, methods, and examples provided herein are illustrative only and not intended to be limiting.

The present disclosure relates to providing at least an ear-wearable device including neuro-bud(s) for monitoring various bio-signals and the method of its operation thereof. The ear-wearable device is implemented to efficiently enhance the signal fidelity for continuous and simultaneous monitoring of various bio-signals related to the subject. The neuro-bud(s) includes a plurality of biosensors, fabricated for tracking various physiological signals through the ear canal and its periphery of the subject, which can further be utilized for analyzing the changes in bio-electrical signals for generating at least health insights on the basis of related physiological parameters.

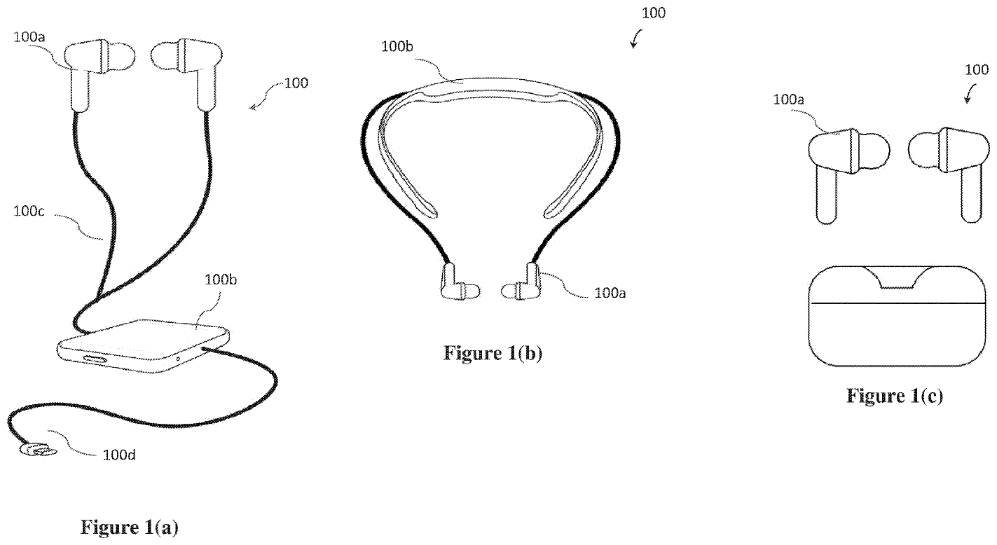

FIG. 1 a illustrates a schematic view of a wired ear-wearable device, in accordance with an embodiment of the present disclosure.

Referring to FIG. 1(a), in an embodiment, the ear wearable device 100 is a wired device including a pair of neuro-buds 100a that may be worn by a user in at least one ear canal region, to capture one or more for detecting at least one physiological parameter of a user during operation. The ear wearable device 100 includes a controller 100b which is adapted to be in communication with the pair of neuro-buds, through a wired connection means 100c. The controller 100b is adapted to further process the determined physiological parameters. The controller 100b is also configured to be programmed within one or more of any of or any combination of software application(s), wearable technology, or interconnected communication systems(s). The ear wearable device 100 is further adapted to be coupled with a User Equipment (UE) through a wire connection 100d for displaying and storing the processed output transmitted from the controller 100b.

FIG. 1(b) illustrates a schematic view of a wireless ear-wearable device, in accordance with an embodiment of the present disclosure.

Referring to FIG. 1(b), in another embodiment, the ear wearable device 100 is adapted to undergo wireless communication with the UE. Further, the ear wearable device 100 includes a controller 100b & a battery backup integrated within the ear wearable device 100.

Referring to FIG. 1(c), in another embodiment, the ear wearable device 100 is adapted to undergo wireless communication with the UE. Further, the ear wearable device 100 has a processing unit & battery backup integrated within the device, and inside the neuro-buds (100a).

Referring to FIG. 1(a), FIG. 1(b), and FIG. 1(c) each pair of neuro-buds may be adapted to be inserted into an ear of a user for detecting at least physiological parameters of the user such as Electroencephalogram (EEG), blood pressure, temperature, etc. The contour of the neuro-bud(s) 100a is adapted to be adjusted at least according to a portion of a canal and a concha region of the user's ear.

The neuro-buds) 100a is adapted to dynamically adjust when inserted inside the user's ear and extend to get positioned in the user's ear, allowing at least the electrophysiological sensor to sense and measure at least the user's electrophysiological signal. The neuro-bud 100a is also adapted for delivering one or more instructions to guide a user at least through an audio signal.

Further, the controller 100b may be adapted for at least conditioning and processing at least the bio-signals received from the neuro-buds 100a and further feeding one or more processed bio-electrical signals to the User Equipment for display, storage, and further analysis.

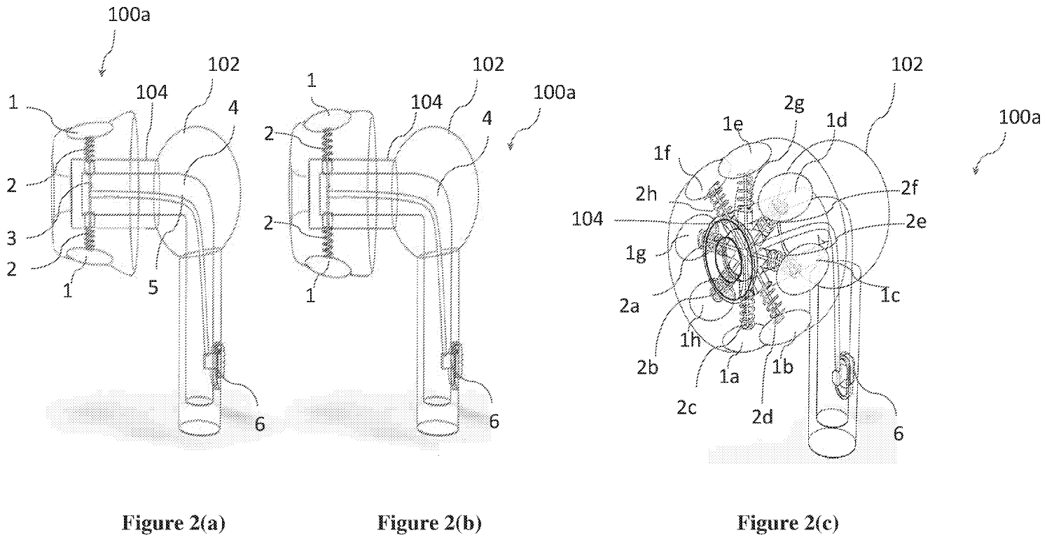

FIG. 2(a), FIG. 2(b), and FIG. 2(c) illustrate schematic views of the neuro-bud 100a, in accordance with an embodiment of the present disclosure.

Referring to FIG. 2(a), FIG. 2(b), and FIG. 2(c), the neuro-bud 100a is designed, preferably, in an earphone shape. The neuro-bud 100a may include, but is not limited to, a housing 102, a hub 104 disposed in the housing 102, a plurality of springs 2, 2a-2h disposed on the hub 104, and a biosensor electrode 1, 1a-1h disposed on each spring 2, 2a-2h, The plurality of springs 2, 2a-2h may be adapted to expand for extending the biosensor electrode 1, 1a-1h to establish contact with the ear canal and to contract for retracting the biosensor electrode 1, 1a-1h to break the contact.

Particularly, FIG. 2(a) illustrates a schematic view of a neuro-bud of the ear-wearable device depicting a plurality of springs in retracted mode, in accordance with an embodiment of the present disclosure.

The retraction mechanism is adapted for dynamically fitting the biosensor electrodes 1, 1a-1h within the ear canal of the user or subject, thereby enabling high signal fidelity. During the retracted state of the biosensor electrodes 1, 1a-1h, the springs 2 are in a compressed state, causing the size of the neuro-buds 100a to reduce.

FIG. 2(b) illustrates a schematic view of the neuro-bud depicting a plurality of springs in a released mode, in accordance with an embodiment of the present disclosure.

When the springs 2, 2a-2h are in the released state, the biosensor electrodes 1, 1a-1h may be adapted to be in contact with an ear canal of the user for detecting at least one of the physiological parameters of the user. Particularly, the size of the neuro-bud 100a along with electrodes 1, 1a-1h expands and touches the lining of the ear canal, when allowed to release inside the ear canal of the user. The plurality of biosensor electrodes 1, 1a-1h may be adapted to monitor at least physiological signals such as neural activity (such as Electroencephalogram (EEG)), cardiac activity monitoring systems (such as (ECG)), muscular activity (such as Electromyography (EMG)), Body Temperature, Skin resistance, Blood Pressure Blood Oxygen Saturation level, perspiration level, electrolyte levels, etc.

In an embodiment, the materials of the biosensor electrode 1, 1a-1h for obtaining the physiological signals may include at least Solid, semi-solid, or flexible forms of Silver/Silver Chloride based or Gold based or other forms of Conductive polymer substrate, including but not limited to ones infused with Carbon Nanotubes or similar conduction enhancing materials. :Further for improved conductivity and long-term monitoring, it can also be based on at least solid, semi-solid, or flexible forms of Graphene-based materials including but not limited to various Graphene Oxides, fabricated using methods like but not limited to, thermal reduction or surface coating.

Referring to FIG. 1 and FIG. 2, the ear-wearable device 100 may also include an actuating switch 6 disposed on the housing 102, a first thread 5 disposed along a thread-guide channel and adapted to couple the actuating switch 6 with the hub 104, and a second thread 3 adapted to connect the first thread with the plurality of springs 2, 2a-2h. The plurality of springs 2, 2a-2h may be adapted to expand and contract based on the actuation of the actuating switch 6 for establishing and breaking the contact, respectively.

Therefore, the neuro-bud 100a includes the plurality of threads 3, 5, which controls the retraction/releasing of the neuro-buds 100a for comfortable wearing within the ear canal thereby suppressing the event of improper fitting of the device 100 inside the ear canal. The thread 5 traverses through a thread-guide tunnel 4 and couples with a switch 6 which is meant for controlling the retraction/releasing of the neuro-buds 100a.

in an embodiment, the plurality of springs 2, 2a-2h may be circumferentially disposed on an outer surface of the hub 104 such that the biosensor electrodes 1, 1a-1h maintain uniform contact with the ear canal. FIG. 2(c) illustrates a schematic perspective view of the neuro-bud, in accordance with an embodiment of the present disclosure.

Referring to FIG. 2(c), the schematic perspective view of the neuro-bud 100a is provided. The neuro-bud 100a of the ear wearable device 100 is coupled with the plurality of biosensor electrodes 1a-1h mounted over the periphery of the neuro-buds 100a for effectively sensing at least the bio-signals from the user's ear canal. Each bio-sensor electrodes 1, 1a-1h is coupled with the thread-guide tunnel 4 at least through the spring 2a-2h which enables the easy and comfortable wearing of the device 100.

The values detected by the neuro-buds may then be transmitted to a controller for further processing.

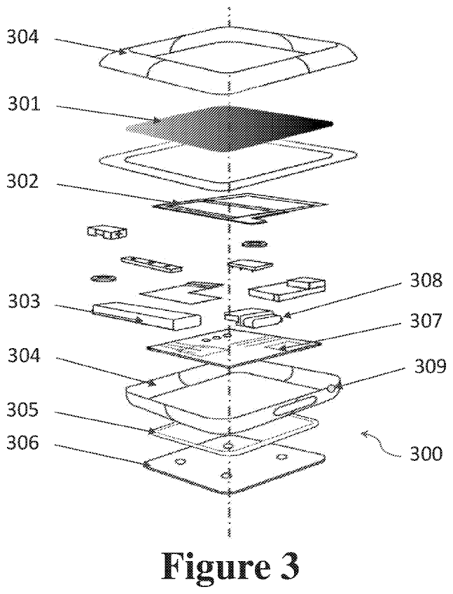

FIG. 3 illustrates the exploded view of a controller 300 of the ear-wearable device, in accordance with an embodiment of the present disclosure, without departing from the scope of the present disclosure.

The controller 300 may be adapted to receive at least one value of the at least one physiological parameter detected by the biosensor electrode 1, 1a-1h, and generate health insights of the user based on the at least one physiological parameter.

The controller 300 may be configured for conditioning and processing at least the physiological signals received from various biosensor electrodes 1, 1a-1h, of the neuro-buds 100a and further feeding at least one or more processed bio-electrical signals to a User Equipment (UE) for display and analysis. The controller 300 includes at least a touch module 301, a display module 302, a rechargeable battery 303, a casing 304, adhesive gasket 305, a back cover glass 306, a circuit board 307, a connection port 308, and an operating switch 309, as also referred in FIG. 3,

The display module 302 can display one or more options related to the operational settings of the ear wearable device. The display module 302 is also adapted to display at least one or more physiological parameters determined by the controller, for example, instant EEG report, and/or ECG report, and/or fitness information can be displayed over the display module of the controller 300.

The touch module 301 and the display module 301 of the controller 300 helps the user in controlling at least one or more settings of the ear-wearable device 100 for operation and displaying at least one value of the at least one physiological parameter detected by the biosensor electrode 1, 1a-1h.

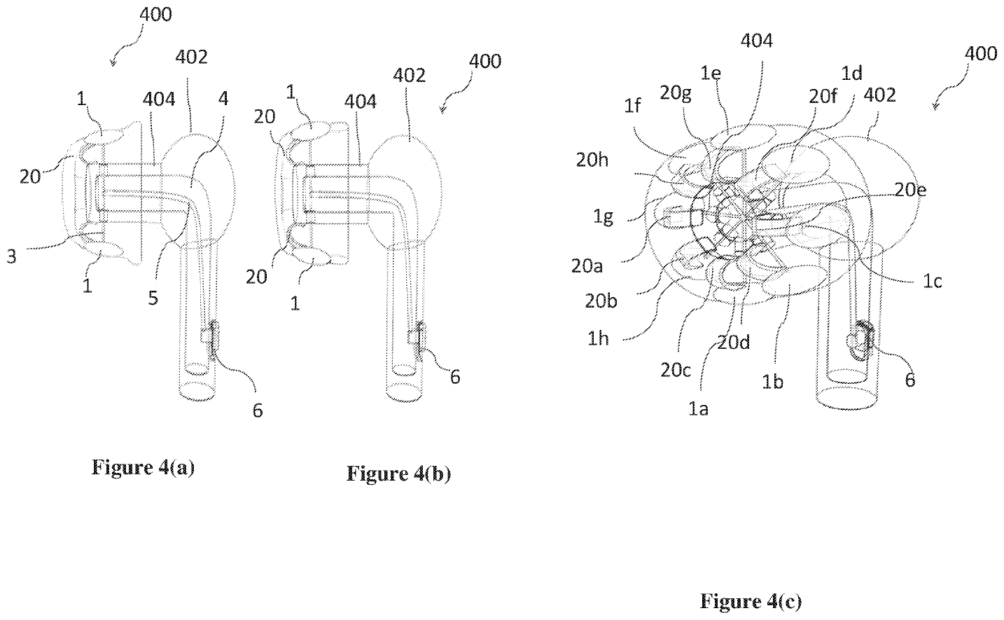

FIG. 4(a), FIG. 4(b), and FIG. 4(c) illustrate schematic views of the neuro-bud 100a, in accordance with an embodiment of the present disclosure.

In another embodiment of the present disclosure, there is provided the neuro-bud 400 which includes a plurality of flexible curved leaves 20, 20a-20h disposed on a huh 404. In the present embodiment, instead of the plurality of springs 2, 2a-2h, the plurality of curved leaves 20, 20a-20h have been used, while the other components and construction remains the same as that of the embodiment disclosed in reference to the FIG. 2.

Being flexible, the curved leaves 20, 20a-20h exhibit retention mechanism. The plurality of flexible curved leaves 20, 20a-20h are adapted to expand for extending the biosensor electrodes 1, 1a-1h to establish contact with the ear canal, and to contract for retracting the biosensor electrode 1, 1a-1h to break the contact.

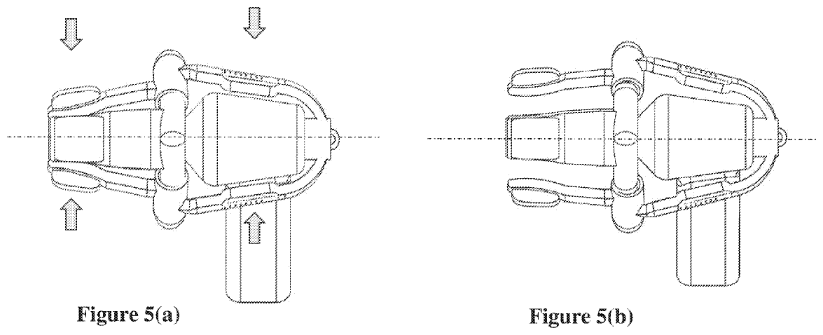

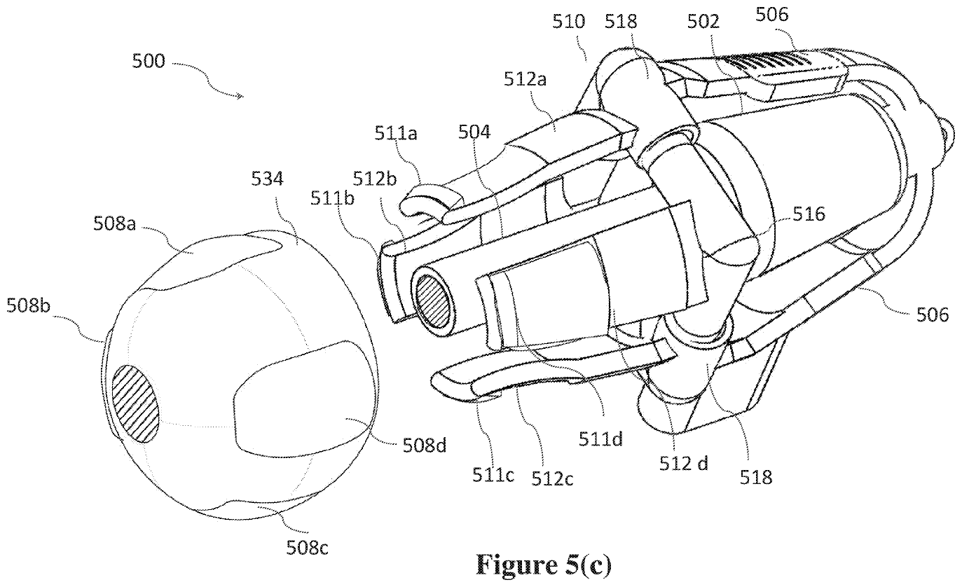

FIG. 5(a) illustrates a schematic diagram of a neuro-bud in a compressed state, in accordance with an embodiment of the present disclosure.

FIG. 5(b) illustrates a schematic diagram of the neuro-bud in an expanded state, in accordance with an embodiment of the present disclosure.

FIG. 5(c) illustrates a schematic perspective view of the neuro-buds, including collapsible member(s), in accordance with an embodiment of the present disclosure.

In another embodiment of the present disclosure, the ear-wearable device 100 may include a plurality of neuro-buds 500. Each neuro-bud 500 may include, but is not limited to, a housing 502, a hub 504 disposed in the housing 502, a plurality of actuating members 506 supported on either side of the housing 502. The plurality of actuating members 506 is provided with spring-like properties on either side of the housing 502. Each neuro-bud 500 further includes a collapsible member assembly 510 supported on the other end of the actuating members 506, an earbud 534, a plurality of connecting arms 512a-512d with projected ends 511a-511d for holding the earbud 534, and a plurality of biosensor electrodes 508a-508d coupled to the earbud 534 that is adapted to be in contact with an ear canal of a user for detecting the at least one physiological parameter of the user, through the retraction and expansion of connecting arms 512a-512d.

The collapsible member assembly 510 of the neuro-bud 500 is adapted for extending the connecting arms 512a-512d which in turn extends the biosensor electrode 508a-508d on the earbud 534 to establish contact with the ear canal and to retract for retracting the connecting arms 512a-512d which in turn retracts biosensor electrode 508a-508d to break the contact, based on actuation of the actuating member 506.

Further, the neuro-bud 500 is provided with a controller 300, which is in communication with the biosensor electrode 508a-508d and is adapted to: receive at least one value of the at least one physiological parameter detected by the biosensor electrode 508a-508d. The controller may be adapted to generate health insights of the user based on the at least one physiological parameter.

Referring to FIG. 5(a), FIG. 5(b), and FIG. 5(c) the neuro-bud 500 may include the collapsible member provided with a mechanism of retraction. A user may control the biosensor electrodes using a press-actuation setup. The biosensor electrodes by default are in the extended state. When the user presses the actuating member, the biosensor electrodes completely retract into the neuro-buds. The user may easily insert the neuro-buds inside the ear canal in the retracted state. On releasing the actuating member, the biosensor electrodes of the neuro-buds may extend out as required after being released. When the actuating members are pressed, the motion is transmitted linearly into the outer collapsible members, which in turn exerts a force on the inner collapsible members to compress and reduce its overall size.

When the actuating member 506 is actuated, the linear movement of the actuating member 506 of the neuro-bud gets translated into contraction of the collapsible member assembly 510 for retracting the biosensor electrodes 508a-508d.

The collapsible member assembly 510 may further include an inner member 516, and an outer member 518 surrounding the inner member 516 and adapted to be supported on the other end of the actuating members 506, wherein the inner member 516 is adapted to move relative to the outer member 518 for expansion and contraction.

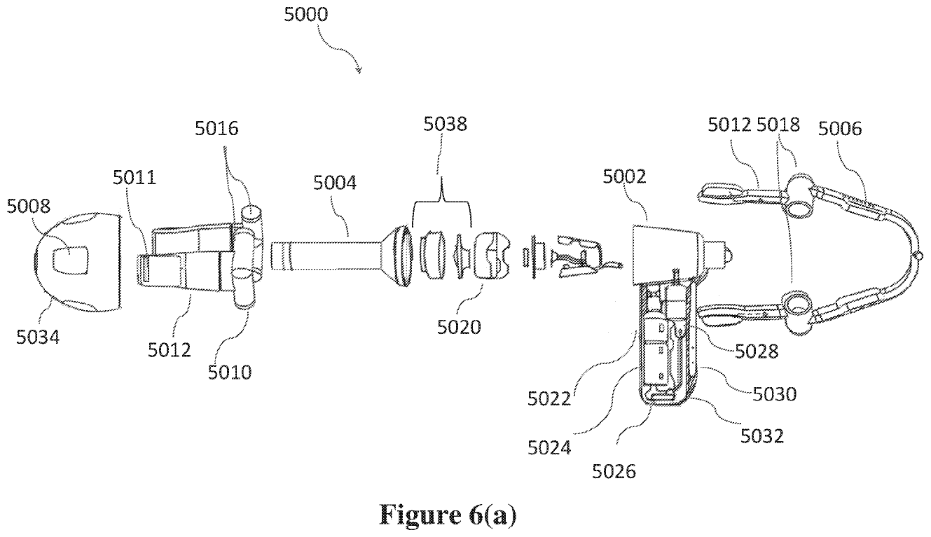

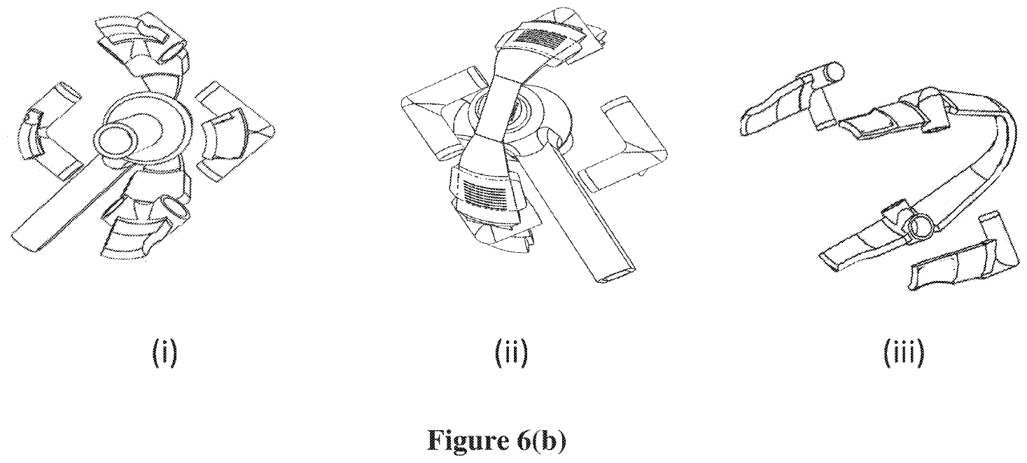

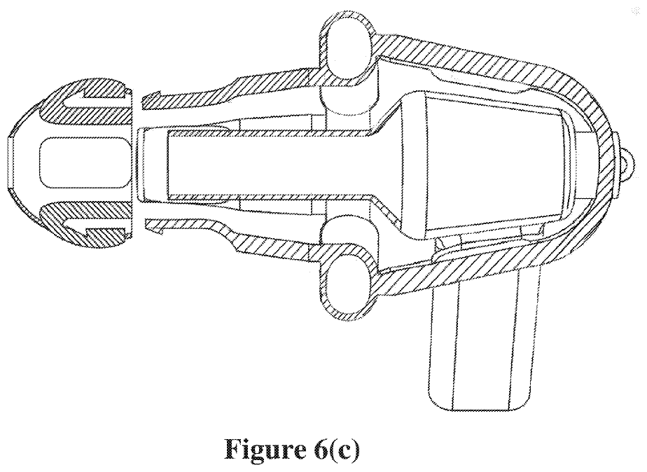

FIG. 6(a) illustrates a schematic exploded view of a neuro-bud 5000, comprising collapsible member(s) 5016, 5018, as referred to in FIG. 5, in accordance with an embodiment of the present disclosure,

In an embodiment, there is provided the neuro-bud 5000 of an ear-wearable device 100 for detecting at least one physiological parameter of a user, the neuro-bud 5000 comprising: a housing 5002, a hub 5004 that is disposed in the housing 5002, a plurality of actuating members 5006 on either side of the housing 5002; a collapsible member assembly 5010 which is further comprising an inner member 5016 and an outer member 5018.

The collapsible member assembly 5010 of the neuro-bud is further supported on the other end of the actuating member 5006. The neuro-bud 5000 is further provided with an earbud 5034 at its proximal end to provide the user comfort in wearing during the use, Preferably, earbud 5034 is made from materials such as, but not limited to, silicon-based or plastic material, flexible polymer, etc. The earbud 5034 comprises a plurality of electrodes 5008 embedded on it which establishes direct contact with the skin of an ear canal for detecting the at least one physiological parameter of the user.

In an embodiment, the earbud 5034 may alternatively function as a complete electrode on its own, if constructed as a fully conductive body.

The plurality of connecting arms 5012 are coupled to the collapsible member assembly 5010 which is fitted inside an electrode housing 5002. The collapsible member assembly 5010 is adapted to expand for extending the connecting arms 5012, which in turn extend the biosensor electrodes 5008 to establish contact with the ear canal and to retract for retracting the connecting arms 5012 which in turn retracts the biosensor electrode 5008 to break the contact, based on actuation of an actuating member 5006.

Further, the neuro-bud 5000 comprises a controller 5020 for processing at least the signals related to physiological parameters and at least a plurality of audio system components such as a microphone 5026, a speaker 5038, and for inputting/outputting sound signals to/from an external device. Further, the neuro-bud 5000 is provided with a stem 5022, which comprises at least a battery 5024 for providing DC supply to the various components of the neuro-bud 5000, a network module 5028, and a charging module 5032.

The stem 5022 is also provided with an indication light 5030 whose intensity is adapted to vary in accordance with at least the brain activity/physiological states such as breathing profile of the user, such that the indication light 5030 may blink frequently or slowly as the brain activity of the user increases or decreases, respectively.

Similarly, the indication light 5030 may, but not limited to, change the color of light to reflect the mental status of the user. For example, the indication light 5030 may, but not limited to, turn green during the meditation phase of the user, or the indication light 5030 may turn red during the critical brain activity/heart rate activity of the user, etc.

FIG. 6(b) illustrates a schematic perspective view of the collapsible member, an actuating member of the neuro-bud, in accordance with an embodiment of the present disclosure.

FIG. 6(c) illustrates a cross-sectional view of the collapsible member, earbud, with at least a locking mechanism, such as a slide-and-lock mechanism, to connect the earbud over the collapsible member. This may enable the user to have the freedom to choose between different electrode materials, sizes, and shapes of the earbud, based on comfort and efficiency required.

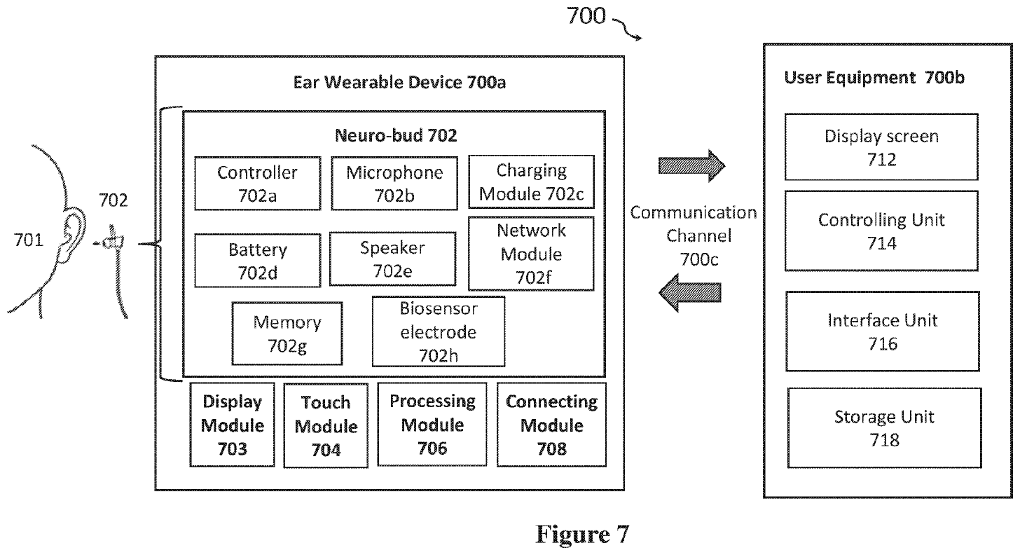

FIG. 7 illustrates the use case scenario 700 of an ear-wearable device 700a in communication with User Equipment (UE) 700b, in accordance with an embodiment of the present disclosure.

In one of the use case scenarios, primarily, a user 701 may connect the ear-wearable device 700a with the User Equipment 700b through a communication channel 700c. The communication channel 700c can either be a wired communication channel or a wireless communication channel. A connecting module 708 is also provided within the ear-wearable device 700a for setting up a wired/wireless connection with the User Equipment 700b through an interface unit 716.

An acquisition unit may optionally be provided to set up the communication of the ear-wearable device 700a with a User Equipment 700b. A Processing Module 706 of the ear-wearable device 700a is coupled at least with the one or more biosensor electrodes 702h of a neuro-bud 702 for processing at least the captured physiological signals under test/monitoring. The user 701 may control one or more settings of the ear-wearable device 700a via a touch module 704, coupled with at least a display module 703, based user interface for operation and displaying of at least one value of the at least one physiological parameter to be detected by the biosensor electrode 702h of the neuro-bud 702.

The neuro-bud 702 may include, but is not limited to, a controller 702a, a microphone 702b, a charging module 702c, a battery 702d, a speaker 702e, a network module 702f, a memory 702g, and at least one or more biosensor electrode 702h for generating health insights of the user 701 based on the at least one physiological parameter, including but not limited to neural activity (EEG), cardiac activity monitoring systems (ECG), muscular activity (EMG), Body Temperature, Blood pressure, and mental stress.

The user may either control and/or visualize output on at least the display module 703 or touch module 704 of the ear wearable device 700a or on a display screen 712 of the User Equipment 700b, where output may also be stored in a storage unit 718 for further use or analysis. The controlling unit 714 of the User Equipment 700b may also be configured to control the operation of the ear wearable device 700a.

In another implementation, a digital phenotyping feature may also be induced with the User Equipment (UE), coupled with the ear-wearable biomedical device, which may help the user in tracking the data captured through the biosensors, and using the biosensors on-board of the User Equipment and thereby analyzing the patterns of usage (Typing speed/Screen Brightness/Screen wake times/number of calls In & Out/No of Notifications, etc.) for generating a complete mapping of the users state, and then deliver results accordingly. The application installed within the User Equipment on receiving the processed biosensor signals from the neuro-buds 100a may display the output over the display module 703. According to the output displayed on the display module, the user may further be recommended with application-based meditation training &. focus exercises. The suggestion is refreshed based on the biofeedback from the user.

In another implementation of the present disclosure, the ear-wearable device 100 may be implemented to efficiently enhance the wearability and portability features while diagnosing/monitoring various physiological signals related to the subject. The ear-wearable device 100 is further implemented to competently interface with a Cloud server independently or at least via connected User Equipment for displaying, analyzing, and storing the monitored signals.

In another implementation, the bio-signals may be broadcasted through communication wire or wirelessly (via WiFi network, or Bluetooth RE connection, or BLE, etc.) from the processor to either to a User Equipment such as mobile phone, smartwatch, PDA, laptop, etc. or a touchscreen-based user interface equipped to transmit/receive/display such signals. The bio-signals such as brain activity, EEG,

ECG, etc. and other motion sensors such as Accelerometer, Gyrosensor, compass, etc. may be transmitted from the processor to the User Equipment or user interface via these or any other currently available communication methods for visual displays or any that may become available in the future.

In another implementation, the ear-wearable device 100 may also be used as a personalized mental and physical fitness coach that can recommend the best routines and at least Meditation practices based on the biofeedback from the user by analyzing the physiological signals and other sensory data.

In another implementation, the ear wearable device 100 may provide a tangible scoring and rating/assessment system that helps the user set goals. Recommendations may he provided on how to achieve the goal, and they may also he able to share it with other users in the system and leaderboard system.

In another implementation, the controller 100b adapted for processing one or more physiological signals at least by filtering and converting Analog Signal to Digital signals, and vice versa, etc. Alternatively, there may be provided a controlling module which performs signal processing like the Fast Fourier Transform (FFT), etc. on conditioned signals to analyze the dominance of brainwaves at different frequencies like Alpha (At 7-12 Hz), theta, etc. or cardiac activity signals (ECG), etc.

The controller 100b may also be adapted for performing stress mapping based on the correlation between the various brainwave signals (alpha, theta, etc.), feedback from the user, and other physiological parameters of the user, to analyze the stress level of the user. Further, the correlation of the stress mapping reading can be engaged as an indicator of the relaxation level of the user, which can be observed over the mobile phone or a computer, etc. as an output, and training recommendations may be suggested.

The controller 100b may be coupled between the neuro-buds 100a and the UE. The controller 100b may process the incoming Analog Signal to Digital signals which may further be transmitted to the User Equipment like smartphones, smartwatches, and laptops, etc.

The controller 100b or processing module or processors, etc., may include an application-specific integrated circuit (ASIC), a chipset, a logic circuit, and/or a data processing device. The memories may include a Read-Only Memory (ROM), a Random Access Memory (RAM), a flash memory, a memory card, a storage medium, and/or another storage device.

The RF units may include a baseband circuit for processing a wireless signal. When an embodiment is embodied as software, the described scheme may be embodied as a module (process, function, or the like) that executes the described function. The module may be stored in a memory, and may be executed by a processor. The memory may be disposed inside or outside the processor and may be connected to the processor through various well-known means.

In another implementation, the ear wearable device 100 may optionally be used for diagnosing medical ailments like Epilepsy and other seizure disorders, sleep disorders, attention disorder, behavioral disorder, developmental delays, etc. The biosensor results obtained from the neuro-buds 100a may be used for diagnosing ailments mentioned above like epileptic seizure traces and predicting them pre-emptively. Similarly, the cardiac data from the ECG module of the neuro-buds 100a may be used to compute the corresponding blood pressure values using but not limited to a pulse transmit time calculation and its logarithmic dependency.

Similarly, the data from neuro-bud 100a may be used in conjunction with a blood glucose monitoring application to monitor and detect sudden changes in the blood sugar levels, for example, conditions like hypoglycemia and give suggestions/alerts accordingly.

Similarly, a temperature sensor coupled with the neuro-bud 100a is meant for actively tracking and plotting the user's body temperature. The added advantage here is that it maps the temperature from the ear canal which is a region affected very little by room temperature.

In another implementation, the housing 102 of neuro-bud 100a may be, but not limited to, adapted for housing one or more sensors including motion sensor such as accelerometer, gyrosensor, compass, etc., and other compact electronics modules like Analog to digital converter, processor, GPS, battery, etc.

In another implementation, the ear wearable device 100, while functioning as an earphone for delivering sound waves, is adapted for tracking the bio-signals of the user in the background passively and generating reports in a periodic timeframe (for example, 1 day/Week/Month) and detect out anomalies compared to the generic patterns of signals of the users using but not limited to Machine Learning techniques.

In another implementation, the data from the ear-wearable device 100 may also be transmitted to the User Equipment, for example, smartphone, PC, laptop, etc., which is further adapted to set-up Internet communication for enabling cloud connectivity which facilitates the doctors and medical practitioners to remotely monitor and analyze the bio-signals. The present disclosure may facilitate the clinicians and doctors of rural areas where such facilities are unavailable.

In view of the aforesaid, there are provided various advantageous applications relating to the present disclosure:
Mood mapping and mood-based music player;
Stress level tracking & fatigue alert system, panic detection;
Meditation and Breathing tracker enabling feedback based guided meditation, goals & scores based guided meditation tracker, mindfulness meditation, and relaxation techniques, etc.
Tracking and helping diagnose pre-emptively medical ailments such as seizure disorders, sleep disorders, attention disorders, behavioral disorders, developmental delays, etc.
Affordable real-time monitoring of health parameters enabling better Rural/remote health care using smartphones;
Sleep tracking, Sleep Quality Monitoring, Sleep Cycle based alarms;
Drowsiness and Fatigue monitoring and alert system especially for night-time drivers, pilots, and Industrial workers, etc.
Specialized and customized monitoring in professionals handling high-risk tasks such as astronauts, pilots, and air travelers, race car drivers, defense personals, police, loco-pilots, etc.
Provide biosignal data like EEG to enable authentication/detection of Users based on their signature biosignal pattern.
Provide biosignal data and other motion sensory data to aid the experience for entertainment/gaming applications including but not limited to Virtual reality applications, Augmented reality applications, etc.

At least by virtue of aforesaid features, the present subject matter at least renders various salient features such as:

The data recorded by the User Equipment can be used to diagnose various ailments or diseases and also help predict them pre-emptively. The monitored data can further be used by Doctors to make better insightful decisions when treating the patient, especially in rural areas where such facilities are unavailable;

Obtaining the biofeedback from the subject through analysis of physiological parameters, and analyzing the sensor data from the phone (digital phenotyping) for generating a complete mapping of the subject's state, and thereby delivering the outcomes relating to but not limited to meditation, mindfulness & focus training.

While specific language has been used to describe the present subject matter, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1. An ear-wearable device (100) comprising:
a plurality of neuro-buds (100a), each neuro-bud (100a) comprising:
a housing (102);
a hub (104) disposed in the housing (102);
a plurality of springs (2, 2a-2h) disposed on the hub (104); and
a biosensor electrode (1, 1a-1h) disposed on each spring (2, 2a-2h) and adapted to be in contact with an ear canal for detecting at least one physiological parameter of a user,
wherein the plurality of springs (2, 2a-2h) is adapted to expand for extending
the biosensor electrode (1, 1a-1h) to establish contact with the ear canal and to contract for retracting the biosensor electrode (1, 1a-1h) to break the contact; and a controller (100b, 300) in communication with the biosensor electrode (1, 1a-1h) and adapted to:
receive at least one value of the at least one physiological parameter detected by the biosensor electrode (1, 1a-1h); and
generate health insights of the user based on the at least one physiological parameter.

2. The ear-wearable device (100) as claimed in claim 1, comprising:
an actuating switch (6) disposed on the housing (102);
a first thread (5) disposed along a thread-guide channel and adapted to couple the actuating switch (6) with the hub (104); and
a second thread (3) adapted to connect the first thread with the plurality of springs (2, 2a-2h),
wherein the plurality of springs (2, 2a-2h) is adapted to expand and contract based on the actuation of the actuating switch (6) for establishing and breaking the contact, respectively.

3. The ear-wearable device (100) as claimed in claim 1, wherein the plurality of springs (2, 2a-2h) is circumferentially disposed on an outer surface of the hub such that the biosensor 30 electrodes (1, 1a-1h) maintain uniform contact with the ear canal.

4. The ear-wearable device (100) as claimed in claim 1, wherein the biosensor electrode (1, 1a-1h) is adapted to sense and analyze at least one physiological parameter comprising at least one of brainwave, heart rate, blood pressure, and a temperature of the user.

5. The ear-wearable device (100) as claimed in claim 1, wherein the biosensor electrode (1, 1a-1h) comprising at least one of solid or semi solid or flexible form of:
Graphene or Graphene Oxide based electrode,
Carbon nanotube based electrode,
Conductive polymer substrate based electrode,
Silver-based/Gold-based electrode, and/or
material with conductive properties.

6. The ear-wearable device (100) as claimed in claim 1, wherein the controller (100b, 300) of the ear-wearable device (100) is provided with at least an interface unit, a touch module (301) and a display module (302), wherein the controller (100b, 300) is adapted for controlling at least one or more settings of the ear-wearable device (100) for operation and 15 displaying at least one value of the at least one physiological parameter detected by the biosensor electrode (1, 1a-1h).

7. A neuro-bud (100a) of an ear-wearable device (100) for detecting at least one physiological parameter of a user, the neuro-bud (100a) comprising:
a housing (102);
a hub (104) disposed in the housing (102);
a plurality of springs (2, 2a-2h) disposed on the hub (104); and
a biosensor electrode (1, 1a-1h) disposed on each spring (2, 2a-2h) and adapted to be in contact with an ear canal for detecting the at least one physiological parameter of the user,
wherein the plurality of springs (2, 2a-2h) is adapted to expand for extending the biosensor electrode (1, 1a-1h) for establishing contact with the ear canal and to contract for retracting the biosensor electrode (1, 1a-1h) for breaking the contact.

8. A neuro-bud (400) of an ear-wearable device (100) for detecting at least one physiological parameter of a user, the neuro-bud (400) comprising:
a housing (402);
a hub (404) disposed in the housing (402);
a plurality of flexible curved leaves (20, 20a-20h) disposed on the hub (404); and
a biosensor electrode (1, 1a-1h) disposed on each leaf (20, 20a-20h) and adapted to be in contact with an ear canal for detecting the at least one physiological parameter of the user,
wherein the plurality of flexible curved leaves (20, 20a-20h) is adapted to expand for extending the biosensor electrode (1, 1a-1h) to establish contact with the ear canal and to contract for retracting the biosensor electrode (1, 1a-1h) to break the contact

9. A neuro-bud (500) of an ear-wearable device (100) for detecting at least one physiological parameter of a user, the neuro-bud (500) comprising:
a housing (502);
a hub (504) disposed in the housing (502);
an actuating member (506) supported on either side of the housing (502);
a collapsible member assembly (510) supported on the other end of the actuating member (506);
an earbud (534);
a plurality of biosensor electrodes (508a-508d) coupled to the earbud (534) and adapted to be in contact with an ear canal for detecting the at least one physiological parameter of the user, and
connecting arms (512a-512d) with projected ends (511a-511d) for holding the earbud (534);
wherein the collapsible member assembly (510) is adapted to:
expand to further extend the connecting arms (512a-512d) and the biosensor electrode (508a-508d) to establish contact with the ear canal, and
retract to further retract the connecting arms (512a-512d) and the biosensor electrode (508a-508d) to break the contact, based on actuation of the actuating members (506).

10. The neuro-bud (500) as claimed in claim 8, wherein the collapsible member assembly (510) comprising:
an inner member (516); and
an outer member (518) surrounding the inner member (516) and adapted to be supported on the other end of the actuating member (506), wherein the inner member (516) is adapted to move relative to the outer member (518) for expansion and contraction.

11. The neuro-bud (500) as claimed in claim 8, wherein the ear bud (534) comprises at least a locking mechanism to connect the earbud (534) over the connecting arms (512) of the collapsible member (510).

12. The neuro-bud as claimed in claim 8, wherein a linear movement of the actuating member (506) translates into contraction of the collapsible member assembly (510) to retract the biosensor electrodes (508a-508d), when the actuating member (506) is actuated.

13. An ear-wearable device (100) comprising:
a plurality of neuro-buds (500), each neuro-bud (500) comprising:
a housing (502);
a hub (504) disposed in the housing (502);
an actuating member(506) supported on either side of the housing (502);
a collapsible member assembly (510) supported on the other end of the actuating members (506);
an earbud (534);
a plurality of biosensor electrodes (508a-508d) coupled to the earbud (534) and adapted to be in contact with an ear canal for detecting the at least one physiological parameter of the user; and
connecting arms (512a-512d) with projected ends (511a-511d) for holding the earbud (534),
wherein the collapsible member assembly (510) is adapted to:
expand to further extend the connecting arms (512a-512d) and the biosensor electrode (508a-508d) to establish contact with the ear canal; and
retract to further retract the connecting arms (512a-512d) and the biosensor electrode (508a-508d) to break the contact, based on actuation of the actuating member (506); and
a controller in communication with the biosensor electrode (508a-508d) and adapted to:
receive at least one value of the at least one physiological parameter detected by the biosensor electrode (508a-508d); and
generate health insights of the user based on the at least one physiological parameter.
